(19) United States
(12) Patent Application Publication
(10) Pub. No.: US 2018/0055345 A1
(43) Pub. Date: Mar. 1, 2018

(54) ENDOSCOPE PROCESSOR

(71) Applicant: OLYMPUS CORPORATION, Tokyo (JP)

(72) Inventor: Masanori SUMIYOSHI, Tokyo (JP)

(73) Assignee: OLYMPUS CORPORATION, Tokyo (JP)

(21) Appl. No.: 15/688,933

(22) Filed: Aug. 29, 2017

(30) Foreign Application Priority Data
Sep. 1, 2016 (JP) 2016-170787

(51) Int. Cl. A61B 1/00; H04N 5/235; A61B 1/07; A61B 1/045

(52) U.S. Cl. CPC A61B 1/00172; H04N 5/2354; H04N 2005/2255; A61B 1/045; A61B 1/07

(57) ABSTRACT

An endoscope processor is used in combination with a scanning type endoscope capable of scanning an object by swinging an optical fiber that transmits illumination light supplied from a light source portion and displacing an irradiation position of the illumination light, and includes: a photodetection portion configured to detect return light from the object irradiated with the illumination light and generate and successively output a photodetection signal according to the detected return light; an image generation portion configured to generate an observation image of the object based on the photodetection signal; and a determination portion configured to determine whether or not the illumination light emitted through the optical fiber is intensively radiated to a minimum area, based on a magnitude of dispersion of brightness in the observation image.

### CROSS REFERENCE TO RELATED APPLICATION

This application claims the benefit of Japanese Application No. 2016-170787 filed in Japan on Sep. 1, 2016, the contents of which are incorporated herein by this reference.

### BACKGROUND OF THE INVENTION

### 1. Field of the Invention

The present invention relates to an endoscope processor, and in particular relates to an endoscope processor used in combination with a scanning type endoscope configured to optically scan an object.

### 2. Description of the Related Art

In an endoscope in a medical field, in order to reduce burdens on a subject, various technologies for narrowing a diameter of an insertion portion to be inserted into a body cavity of the subject have been proposed. Then, as one example of such technologies, a scanning type endoscope not including a solid-state image pickup device at a part corresponding to the insertion portion described above is known.

More specifically, a system including the scanning type endoscope is configured, for example, to transmit illumination light emitted from a light source by an optical fiber for illumination, two-dimensionally scan an object in a predetermined scanning route by driving an actuator for swinging a distal end portion of the optical fiber for illumination, receive return light from the object by an optical fiber for light reception, and generate an image of the object based on the return light received by the optical fiber for light reception. Then, for example, Japanese Patent No. 5490340 discloses an endoscope system similar to such a configuration.

More specifically, Japanese Patent No. 5490340 discloses an endoscope system configured to scan a surface of an object by a spiral scanning pattern by swinging an end portion on a light emission side of a fiber for illumination that transmits illumination light supplied from a light source unit. In addition, Japanese Patent No. 5490340 discloses a configuration of detecting whether or not an emission state of the illumination light emitted through the fiber for illumination is abnormal within a period during which the fiber for illumination is swung to an outermost periphery of the spiral scanning pattern.

### SUMMARY OF THE INVENTION

An endoscope processor of one aspect of the present invention is an endoscope processor used in combination with a scanning type endoscope capable of scanning an object by swinging an optical fiber that transmits illumination light supplied from a light source portion and displacing an irradiation position of the illumination light, and includes: a photodetection portion configured to detect return light from the object irradiated with the illumination light and generate and successively output a photodetection signal according to the detected return light; an image generation portion configured to generate an observation image of the object based on the photodetection signal; and a determination portion configured to determine whether or not the illumination light emitted through the optical fiber is intensively radiated to a minimum area, based on a magnitude of dispersion of brightness in the observation image.

### BRIEF DESCRIPTION OF THE DRAWINGS

FIG. 1 is a diagram illustrating a configuration of a main portion of an endoscope system including an endoscope processor relating to an embodiment;

FIG. 2 is a sectional view for describing a configuration of an actuator portion;

FIG. 3 is a diagram illustrating one example of a signal waveform of a drive signal supplied to the actuator portion;

FIG. 4 is a diagram illustrating one example of a spiral scanning route from a center point A to an outermost point B;

FIG. 5 is a diagram illustrating one example of a spiral scanning route from the outermost point B to the center point A;

FIG. 6 is a diagram for describing one example of a specific configuration of an image processing portion;

FIG. 7 is a diagram illustrating one example of a brightness detection area set by a brightness detection portion;

FIG. 8 is a diagram illustrating one example of the brightness detection area set by the brightness detection portion;

FIG. 9 is a diagram illustrating one example of the brightness detection area set by the brightness detection portion; and

FIG. 10 is a diagram illustrating one example of the brightness detection area set by the brightness detection portion.

### DETAILED DESCRIPTION OF THE PREFERRED EMBODIMENT

Hereinafter, the embodiment of the present invention will be described with reference to the drawings.

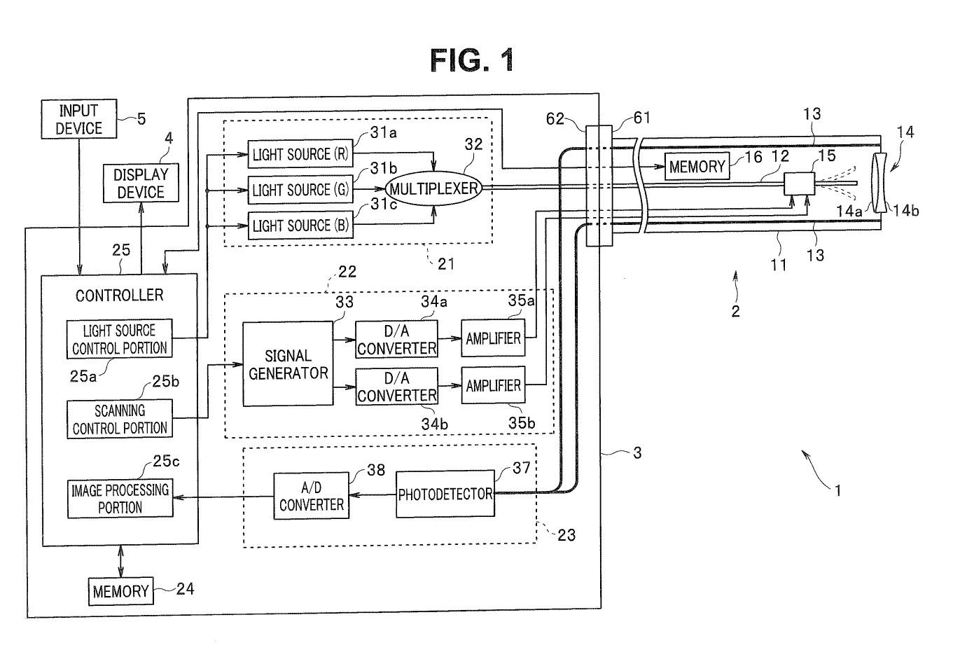

FIG. 1 to FIG. 10 relate to the embodiment of the present invention. An endoscope system 1 is configured, for example, as illustrated in FIG. 1, including a scanning type endoscope (abbreviated simply as an endoscope, hereinafter) 2 to be inserted into a body cavity of a subject, a main body device 3 to which the endoscope 2 can be attachably and detachably connected, a display device 4 connected to the main body device 3, and an input device 5 capable of inputting information and giving an instruction to the main body device 3. FIG. 1 is a diagram illustrating a configuration of a main portion of the endoscope system including the endoscope processor relating to the embodiment.

The endoscope 2 is configured including an insertion portion 11 formed having an elongated shape insertable into a body cavity of a subject.

On a proximal end portion of the insertion portion 11, a connector portion 61 for attachably and detachably connecting the endoscope 2 to a connector receiving portion 62 of the main body device 3 is provided.

Inside the connector portion 61 and the connector receiving portion 62, though not shown in the figure, an electric connector device for electrically connecting the endoscope 2 and the main body device 3 is provided. In addition, inside the connector portion 61 and the connector receiving portion 62, though not shown in the figure, an optical connector device for optically connecting the endoscope 2 and the main body device 3 is provided.

To a part from the proximal end portion to a distal end portion inside the insertion portion 11, a fiber 12 for illumination which is an optical fiber configured to guide illumination light supplied from a light source unit 21 of the main body device 3 and emit the illumination light from an emission end portion, and a fiber 13 for light reception including one or more optical fibers for receiving return light from an object and guiding the return light to a detection unit 23 of the main body device 3 are inserted respectively.

An incident end portion including a light incident surface of the fiber 12 for illumination is arranged at a multiplexer 32 provided inside the main body device 3. In addition, the emission end portion including a light emission surface of the fiber 12 for illumination is arranged near a light incident surface of a lens 14a provided on the distal end portion of the insertion portion 11.

An incident end portion including a light incident surface of the fiber 13 for light reception is fixed and arranged around a light emission surface of a lens 14b on a distal end face of the distal end portion of the insertion portion 11. In addition, an emission end portion including a light emission surface of the fiber 13 for light reception is arranged at a photodetector 37 provided inside the main body device 3.

An illumination optical system 14 is configured including the lens 14a on which the illumination light through the light emission surface of the fiber 12 for illumination is made incident, and the lens 14b that emits the illumination light through the lens 14a to the object.

In a middle portion of the fiber 12 for illumination on a distal end portion side of the insertion portion 11, an actuator portion 15 driven based on a drive signal supplied from a driver unit 22 of the main body device 3 is provided.

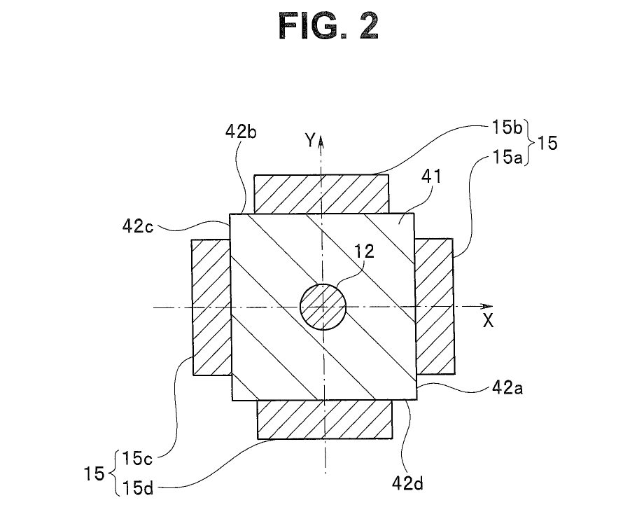

The fiber 12 for illumination and the actuator portion 15 are arranged respectively so as to have a position relation illustrated in FIG. 2, for example on a cross section vertical to a longitudinal axial direction of the insertion portion 11. FIG. 2 is a sectional view for describing a configuration of the actuator portion.

Between the fiber 12 for illumination and the actuator portion 15, as illustrated in FIG. 2, a ferrule 41 as a bonding member is arranged. More specifically, the ferrule 41 is formed by zirconia (ceramic) or nickel, for example.

The ferrule 41 is, as illustrated in FIG. 2, formed as a square pole, and includes side faces 42a and 42c vertical to an X axis direction which is a first axial direction orthogonal to the longitudinal axial direction of the insertion portion 11, and side faces 42b and 42d vertical to a Y axis direction which is a second axial direction orthogonal to the longitudinal axial direction of the insertion portion 11. In addition, at a center of the ferrule 41, the fiber 12 for illumination is fixed and arranged.

The actuator portion 15 includes, for example, as illustrated in FIG. 2, a piezoelectric element 15a arranged along the side face 42a, a piezoelectric element 15b arranged along the side face 42b, a piezoelectric element 15c arranged along the side face 42c, and a piezoelectric element 15d arranged along the side face 42d.

The piezoelectric elements 15a to 15d have polarization directions individually set beforehand, and are configured to expand and contract respectively according to a drive voltage applied by the drive signal supplied from the main body device 3.

That is, the piezoelectric elements 15a and 15c of the actuator portion 15 are configured as an actuator for an X axis capable of swinging the fiber 12 for illumination in the X axis direction by vibrating according to the drive signal supplied from the main body device 3. Furthermore, the piezoelectric elements 15b and 15d of the actuator portion 15 are configured as an actuator for a Y axis capable of swinging the fiber 12 for illumination in the Y axis direction by vibrating according to the drive signal supplied from the main body device 3.

Inside the insertion portion 11, a nonvolatile memory 16 that stores intrinsic endoscope information for each endoscope 2 is provided. Then, the endoscope information stored in the memory 16 is read by a controller 25 of the main body device 3 when the connector portion 61 of the endoscope 2 and the connector receiving portion 62 of the main body device 3 are connected and a power source of the main body device 3 is turned on.

The main body device 3 is configured having a function as the endoscope processor. More specifically, the main body device 3 is configured including the light source unit 21, the driver unit 22, the detection unit 23, a memory 24, and the controller 25.

The light source unit 21 is configured including a light source 31a, a light source 31b, a light source 31c, and the multiplexer 32.

The light source 31a includes a laser light source for example, and is configured to emit light of a red wavelength band (also called R light, hereinafter) to the multiplexer 32 when the light is emitted by control of the controller 25.

The light source 3 lb includes a laser light source for example, and is configured to emit light of a green wavelength band (also called G light, hereinafter) to the multiplexer 32 when the light is emitted by the control of the controller 25.

The light source 31c includes a laser light source for example, and is configured to emit light of a blue wavelength band (also called B light, hereinafter) to the multiplexer 32 when the light is emitted by the control of the controller 25.

The multiplexer 32 is configured to multiplex the R light emitted from the light source 31a, the G light emitted from the light source 31b, and the B light emitted from the light source 31c, and supply the light to the light incident surface of the fiber 12 for illumination.

The driver unit 22 is configured to generate and supply a drive signal DA for driving the actuator for the X axis of the actuator portion 15 based on the control of the controller 25. In addition, the driver unit 22 is configured to generate and supply a drive signal DB for driving the actuator for the Y axis of the actuator portion 15 based on the control of the controller 25. Furthermore, the driver unit 22 is configured including a signal generator 33, D/A converters 34a and 34b, and amplifiers 35a and 35b.

The signal generator 33 is configured to generate a signal having a waveform indicated by an equation (1) below, for example, as a first drive control signal for swinging the emission end portion of the fiber 12 for illumination in the X axis direction and output the signal to the D/A converter 34a, based on the control of the controller 25. Note that, in the equation (1) below, X(t) denotes a signal level at time t, Ax denotes an amplitude value independent of the time t, and G(t) denotes a predetermined function used in modulation of a sine wave sin(2πft).

X(t)=Ax×G(t)×sin(2πft) (1)

In addition, the signal generator 33 is configured to generate a signal having a waveform indicated by an equation (2) below, for example, as a second drive control signal for swinging the emission end portion of the fiber 12 for illumination in the Y axis direction and output the signal to the D/A converter 34b, based on the control of the controller 25. Note that, in the equation (2) below, Y(t) denotes the signal level at the time t, Ay denotes the amplitude value independent of the time t, G(t) denotes a predetermined function used in modulation of a sine wave sin(2πft+4), and φ denotes a phase.

Y(t)=Ay×G(t)×sin(2πft+4)) (2)

The D/A converter 34a is configured to convert the digital first drive control signal outputted from the signal generator 33 to an analog drive signal DA and output the drive signal DA to the amplifier 35a.

The D/A converter 34b is configured to convert the digital second drive control signal outputted from the signal generator 33 to an analog drive signal DB and output the drive signal DB to the amplifier 35b.

The amplifier 35a is configured to amplify the drive signal DA outputted from the D/A converter 34a and output the amplified drive signal DA to the piezoelectric elements 15a and 15c of the actuator portion 15.

The amplifier 35b is configured to amplify the drive signal DB outputted from the D/A converter 34b and output the amplified drive signal DB to the piezoelectric elements 15b and 15d of the actuator portion 15.

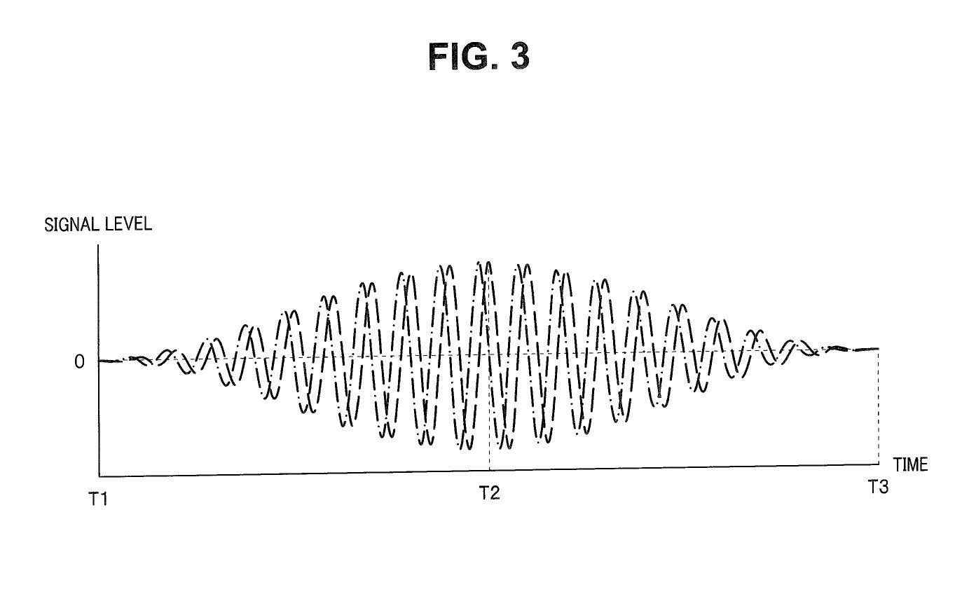

Here, for example, in the above-described equations (1) and (2), in a case that Ax=Ay and φ=π/2 are set, the drive voltage according to the drive signal DA having the signal waveform as illustrated by a broken line in FIG. 3 is applied to the piezoelectric elements 15a and 15c of the actuator portion 15, and the drive voltage according to the drive signal DB having the signal waveform as illustrated by a dashed line in FIG. 3 is applied to the piezoelectric elements 15b and 15d of the actuator portion 15. FIG. 3 is a diagram illustrating one example of the signal waveform of the drive signal supplied to the actuator portion.

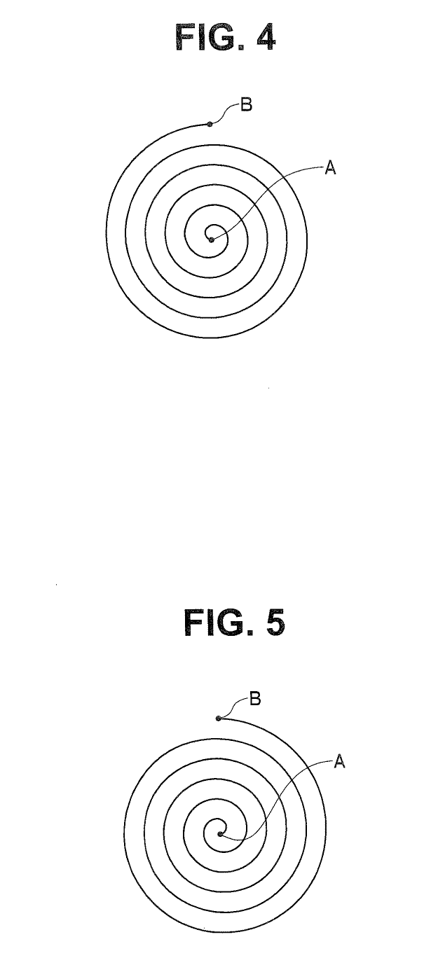

In addition, for example, in the case that the drive voltage according to the drive signal DA having the signal waveform as illustrated by the broken line in FIG. 3 is applied to the piezoelectric elements 15a and 15c of the actuator portion 15 and the drive voltage according to the drive signal DB having the signal waveform as illustrated by the dashed line in FIG. 3 is applied to the piezoelectric elements 15b and 15d of the actuator portion 15, the emission end portion of the fiber 12 for illumination is spirally swung, and a surface of the object is scanned along a spiral scanning route as illustrated in FIG. 4 and FIG. 5 according to such swinging. FIG. 4 is a diagram illustrating one example of the spiral scanning route from a center point A to an outermost point B. FIG. 5 is a diagram illustrating one example of the spiral scanning route from the outermost point B to the center point A.

More specifically, first, at time T1, the illumination light is radiated to a position corresponding to the center point A of the irradiation position of the illumination light on the surface of the object. Thereafter, as the signal level of the drive signals DA and DB increases from the time T1 to time T2, the irradiation position of the illumination light on the surface of the object is displaced to draw a first spiral scanning route to an outer side with the center point A as an origin, and further, when the time T2 comes, the illumination light is radiated to the outermost point B of the irradiation position of the illumination light on the surface of the object. Then, as the signal level of the drive signals DA and DB decreases from the time T2 to time T3, the irradiation position of the illumination light on the surface of the object is displaced to draw a second spiral scanning route to an inner side with the outermost point B as the origin, and further, when the time T3 comes, the illumination light is radiated to the center point A on the surface of the object.

That is, the actuator portion 15 includes the configuration capable of displacing the irradiation position of the illumination light emitted through the emission end portion to the object along the spiral scanning route illustrated in FIG. 4 and FIG. 5 by swinging the emission end portion of the fiber 12 for illumination based on the drive signals DA and DB supplied from the driver unit 22. In addition, the endoscope 2 includes the configuration capable of scanning the object by displacing the irradiation position of the illumination light to be radiated to the object.

The detection unit 23 has a function as a photodetection portion, and is configured to detect the return light received by the fiber 13 for light reception of the endoscope 2, and generate and successively output a photodetection signal according to intensity of the detected return light. More specifically, the detection unit 23 is configured including the photodetector 37, and an A/D converter 38.

The photodetector 37 includes an avalanche photodiode for example, and is configured to detect the light (return light) emitted from the light emission surface of the fiber 13 for light reception, generate an analog photodetection signal according to the intensity of the detected light, and successively output the signal to the A/D converter 38.

The A/D converter 38 is configured to convert the analog photodetection signal outputted from the photodetector 37 to a digital photodetection signal and successively output the signal to the controller 25.

In the memory 24, as control information used when controlling the main body device 3, for example, information of a parameter for specifying the signal waveform in FIG. 3, and a mapping table which is a table indicating a correspondence relation between output timing of the photodetection signal successively outputted from the detection unit 23 and a pixel position to be an application destination of pixel information obtained by converting the photodetection signal is stored. In addition, the control information stored in the memory 24 includes information indicating a brightness detection area used in processing to be described later.

The controller 25 includes an integrated circuit such as an FPGA (field programmable gate array), and is configured to perform an operation according to an operation of the input device 5. In addition, the controller 25 is configured to detect whether or not the insertion portion 11 is electrically connected to the main body device 3 by detecting a connection state of the connector portion 61 in the connector receiving portion 62 through a signal line or the like not shown in the figure. Furthermore, the controller 25 is configured to read the control information stored in the memory 24 when the power source of the main body device 3 is turned on and perform the operation according to the read control information. In addition, the controller 25 is configured including a light source control portion 25a, a scanning control portion 25b, and an image processing portion 25c.

The light source control portion 25a is configured to perform the control for causing the R light, the G light and/or the B light to be emitted from the light source unit 21 as the illumination light based on the control information read from the memory 24. More specifically, the light source control portion 25a is configured to, for example, set light quantities of the R light, the G light and the B light, and perform the control for causing the R light, the G light and the B light of the set light quantities to be successively emitted as the illumination light to the light source unit 21, based on the control information read from the memory 24. In addition, the light source control portion 25a is configured to perform an operation according to a judgement result obtained by a determination portion 53 (to be described later) of the image processing portion 25c.

The scanning control portion 25b is configured to perform the control for causing drive signals for driving the actuator portion 15 to be generated to the driver unit 22, based on the control information read from the memory 24. More specifically, the scanning control portion 25b is configured to perform the control for causing the drive signals DA and DB having the signal waveform as illustrated in FIG. 3 to be generated to the driver unit 22, for example, based on the control information read from the memory 24.

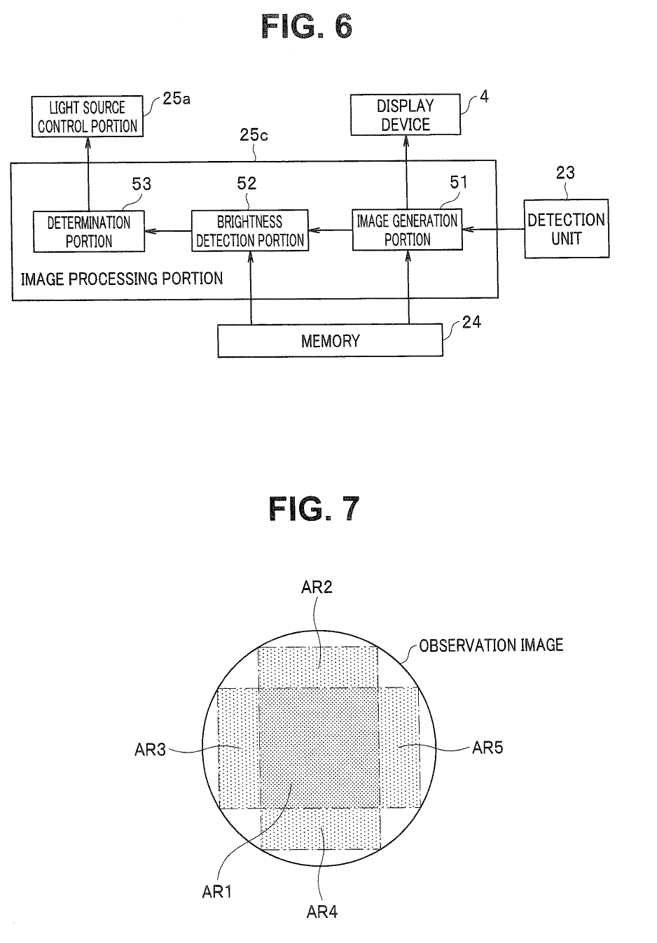

The image processing portion 25c is configured including, for example, as illustrated in FIG. 6, an image generation portion 51, a brightness detection portion 52, and the determination portion 53. FIG. 6 is a diagram for describing one example of a specific configuration of the image processing portion.

The image generation portion 51 is configured to generate an observation image of the object for each frame, and successively output the generated observation image to the display device 4 and the brightness detection portion 52, by converting the photodetection signal successively outputted from the detection unit 23 within a period from the time T1 to T2 to the pixel information and mapping the pixel information, for example, based on the mapping table included in the control information read from the memory 24.

The brightness detection portion 52 is configured to set the plurality of brightness detection areas in the observation image outputted from the image generation portion 51, and acquire a plurality of brightness detection values corresponding to each of the plurality of set brightness detection areas, based on the control information read from the memory 24. In addition, the brightness detection portion 52 is configured to output the plurality of brightness detection values obtained as described above to the determination portion 53.

The determination portion 53 is configured to calculate a brightness dispersion amount which is a value indicating a magnitude of dispersion of brightness in the observation image generated by the image generation portion 51, based on the plurality of brightness detection values outputted from the brightness detection portion 52. In addition, the determination portion 53 is configured to acquire the judgement result by determining the magnitude of the brightness dispersion amount calculated as described above, and output the acquired judgement result to the light source control portion 25a.

The display device 4 includes an LCD (liquid crystal display) for example, and is configured to display the observation image outputted from the main body device 3.

The input device 5 is configured including one or more switches and/or buttons capable of instructing the controller 25 according to the operation by a user. Note that the input device 5 may be configured as a device separate from the main body device 3, or may be configured as an interface integrated with the main body device 3.

Next, the operation or the like of the endoscope system 1 including the configuration as described above will be described.

After connecting the respective portions of the endoscope system 1 and turning on the power source, a user such as an operator gives the instruction for starting scanning of the object to the controller 25 by operating a predetermined switch of the input device 5. Then, according to such an operation of the user, for example, the control for causing the R light, the G light and the B light of a light quantity AL1 to be successively emitted as the illumination light is performed by the light source control portion 25a, the control for swinging the emission end portion of the fiber 12 for illumination so as to draw the spiral scanning route is performed by the scanning control portion 25b, the object is scanned by the illumination light emitted through the emission end portion, the return light from the object is made incident on the detection unit 23 through the fiber 13 for light reception, and the photodetection signal according to the intensity of the return light is outputted from the detection unit 23 to the image generation portion 51.

The image generation portion 51 generates a circular observation image for each frame based on the mapping table included in the control information read from the memory 24, and successively outputs the generated circular observation image to the display device 4 and the brightness detection portion 52.

The brightness detection portion 52 sets five brightness detection areas AR1 to AR5 in the circular observation image for one frame outputted from the image generation portion 51, as illustrated in FIG. 7, for example, based on the control information read from the memory 24. FIG. 7 is a diagram illustrating one example of the brightness detection area set by the brightness detection portion.

More specifically, the brightness detection area AR1 is set as a square area centering on a center pixel of the circular observation image outputted from the image generation portion 51 and positioned more on an inner side than an outermost periphery of the circular observation image, as illustrated in FIG. 7. In addition, the brightness detection areas AR2 to AR5 are set respectively as a rectangular area including one of four sides of the brightness detection area AR1 and being in contact with the outermost periphery of the circular observation image outputted from the image generation portion 51, as illustrated in FIG. 7.

The brightness detection portion 52 performs an arithmetic operation for calculating the brightness detection value corresponding to each of the five brightness detection areas AR1 to AR5, and outputs the five brightness detection values obtained through the arithmetic operation to the determination portion 53.

More specifically, the brightness detection portion 52 calculates an average luminance value AV1 which is an average of luminance values of respective pixels included in the brightness detection area AR1, for example, as the brightness detection value of the brightness detection area AR1. In addition, the brightness detection portion 52 calculates an average luminance value AV2 which is the average of the luminance values of the respective pixels included in the brightness detection area AR2, for example, as the brightness detection value of the brightness detection area AR2. Furthermore, the brightness detection portion 52 calculates an average luminance value AV3 which is the average of the luminance values of the respective pixels included in the brightness detection area AR3, for example, as the brightness detection value of the brightness detection area AR3. In addition, the brightness detection portion 52 calculates an average luminance value AV4 which is the average of the luminance values of the respective pixels included in the brightness detection area AR4, for example, as the brightness detection value of the brightness detection area AR4. Further, the brightness detection portion 52 calculates an average luminance value AV5 which is the average of the luminance values of the respective pixels included in the brightness detection area ARS, for example, as the brightness detection value of the brightness detection area ARS. Then, the brightness detection portion 52 outputs the average luminance values AV1 to AV5 obtained as the brightness detection values of the brightness detection areas AR1 to ARS to the determination portion 53.

The determination portion 53 calculates the brightness dispersion amount based on the five brightness detection values outputted from the brightness detection portion 52, acquires the judgement result by determining the magnitude of the calculated brightness dispersion amount, and outputs the acquired judgement result to the light source control portion 25a.

More specifically, the determination portion 53 calculates variance VA of the average luminance values AV1 to AV5 outputted from the brightness detection portion 52 as the brightness dispersion amount, for example. Then, in the case of detecting that the value of the variance VA is smaller than a predetermined threshold THA, the determination portion 53 acquires the judgement result that the dispersion of the brightness in the observation image generated by the image generation portion 51 is small, and outputs the acquired judgement result to the light source control portion 25a. In addition, in the case of detecting that the value of the variance VA is equal to or larger than the predetermined threshold THA, the determination portion 53 acquires the judgement result that the dispersion of the brightness in the observation image generated by the image generation portion 51 is large, and outputs the acquired judgement result to the light source control portion 25a.

Here, for example, in the case that all the piezoelectric elements 15a to 15d of the endoscope 2 fail, due to stoppage of swinging of the emission end portion of the fiber 12 for illumination by the actuator portion 15, a situation that the illumination light emitted through the emission end portion is intensively radiated to a minimum area near the center point A of the spiral scanning route and the brightness of the minimum area is reflected as the brightness of an entire image area may occur.

In contrast, according to the operation of the determination portion 53 as described above, based on the magnitude of the value of the variance VA, whether or not the illumination light emitted through the emission end portion of the fiber 12 for illumination is intensively radiated to the minimum area outside the endoscope 2 is determined. In addition, according to the operation of the determination portion 53 as described above, when it is detected that the value of the variance VA is smaller than the predetermined threshold THA, the judgement result indicating that the illumination light emitted through the emission end portion of the fiber 12 for illumination is intensively radiated to the minimum area outside the endoscope 2 is acquired. Furthermore, according to the operation of the determination portion 53 as described above, when it is detected that the value of the variance VA is equal to or larger than the predetermined threshold THA, the judgement result indicating that the illumination light emitted through the emission end portion of the fiber 12 for illumination is normally radiated to the object outside the endoscope 2 is acquired.

The light source control portion 25a performs the control for causing the light quantity of the illumination light emitted from the light source unit 21 to be maintained at the light quantity AL1 (present light quantity), in the case of detecting that the dispersion of the brightness in the observation image generated by the image generation portion 51 is large, for example, based on the judgement result outputted from the determination portion 53. In addition, the light source control portion 25a performs the control for causing the light quantity of the illumination light emitted from the light source unit 21 to be lowered to a light quantity AL2 lower than the light quantity AL1 (present light quantity), in the case of detecting that the dispersion of the brightness in the observation image generated by the image generation portion 51 is small, for example, based on the judgement result outputted from the determination portion 53.

Note that the light quantity AL1 is the light quantity corresponding to a class 3R in a standard determining a safety standard of a laser product, for example, and is set as the light quantity of the magnitude suitable for observation inside a body cavity, while being accompanied by a risk in front view of a laser beam. In addition, the light quantity AL2 is the light quantity corresponding to a class 2 in the standard determining the safety standard of the laser product, for example, and is set as the light quantity of the magnitude from which eyes are sufficiently protected by disliking reaction such as blinking, and with which at least the minimum observation inside the body cavity can be performed.

As described above, according to the present embodiment, whether or not the illumination light emitted through the emission end portion of the fiber 12 for illumination is intensively radiated to the minimum area outside the endoscope 2 can be deteimined based on the magnitude of the dispersion of the brightness in the observation image generated by the image generation portion 51. Therefore, according to the present embodiment, presence/absence of abnormality of an emission state of the illumination light emitted from the endoscope 2 can be easily detected without providing a special structure in the endoscope 2, for example.

Note that, according to the present embodiment, for example, when the judgement result indicating that the illumination light emitted through the emission end portion of the fiber 12 for illumination is intensively radiated to the minimum area outside the endoscope 2 is obtained, an operation for generating and outputting visual information and/or sound information capable of reporting the judgement result to a user may be performed in the main body device 3 (controller 25).

In addition, according to the present embodiment, for example, when the judgement result that the dispersion of the brightness in the observation image generated by the image generation portion 51 is large is obtained, light adjusting control of adjusting the light quantity of the illumination light emitted from the light source unit 21 according to the magnitude of the average luminance values AV1 to AV5 may be performed by the light source control portion 25a.

Furthermore, according to the present embodiment, the control for lowering the light quantity of the illumination light emitted from the light source unit 21 from the light quantity AL1 to the light quantity AL2 may be performed not only when the judgement result that the dispersion of the brightness in the observation image generated by the image generation portion 51 is small is obtained once but also when the judgement result is obtained consecutively for a plurality of times, for example. That is, according to the present embodiment, when the observation image in which the dispersion of the brightness is small (the value of the variance VA is smaller than the predetermined threshold THA) is generated consecutively for the plurality of frames by the image generation portion 51, it may be detected that the illumination light emitted through the emission end portion of the fiber 12 for illumination is intensively radiated to the minimum area outside the endoscope 2.

In addition, according to the present embodiment, for example, the determination portion 53 may determine whether or not the illumination light emitted through the emission end portion of the fiber 12 for illumination is intensively radiated to the minimum area outside the endoscope 2, based on the magnitude of a current of the drive signal supplied from the driver unit 22 to the actuator portion 15 and the magnitude of the value of the variance VA. More specifically, for example, the determination portion 53 may obtain the judgement result indicating that the illumination light emitted through the emission end portion of the fiber 12 for illumination is intensively radiated to the minimum area outside the endoscope 2, in the case of detecting that the magnitude of the current of the drive signal supplied from the driver unit 22 to the actuator portion 15 does not fluctuate from a specific magnitude and the value of the variance VA is smaller than the predetermined threshold THA together. Then, according to such a configuration, for example, the situation that an extremely bright or extremely dark observation image is generated by the image generation portion 51 and the situation that the illumination light emitted through the emission end portion of the fiber 12 for illumination is intensively radiated to the minimum area outside the endo scope 2 can be distinguished and detected.

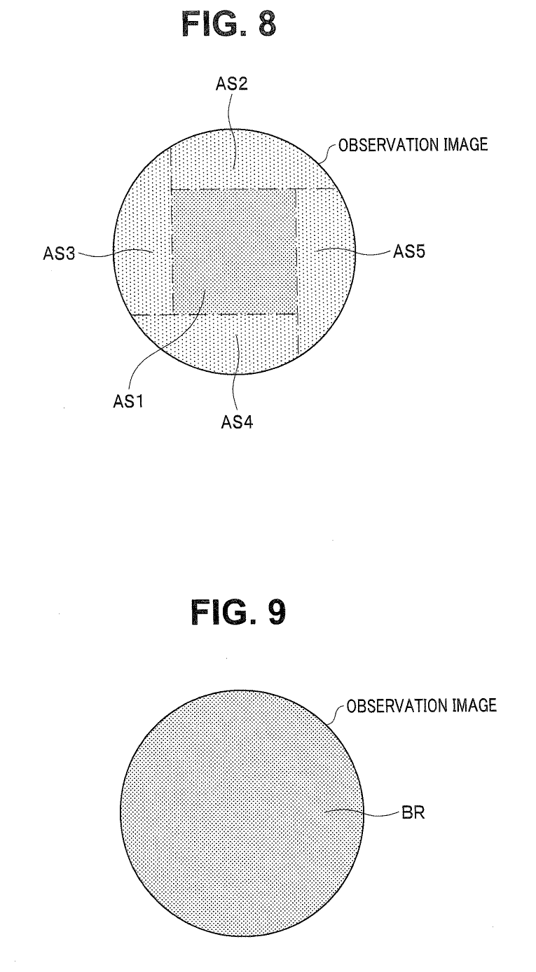

Furthermore, according to the present embodiment, the brightness detection portion 52 may set not only the quadrangular brightness detection areas AR1 to ARS as illustrated in FIG. 7 but also brightness detection areas AS1 to AS5 as illustrated in FIG. 8, for example. FIG. 8 is a diagram illustrating one example of the brightness detection area set by the brightness detection portion.

More specifically, the brightness detection area AS1 is set as a square area centering on the center pixel of the circular observation image outputted from the image generation portion 51 and positioned more on the inner side than the outermost periphery of the circular observation image, as illustrated in FIG. 8. In addition, the brightness detection areas AS2 to AS5 are set respectively as an area for which the remaining area other than the brightness detection area AS1 in the circular observation image outputted from the image generation portion 51 is quadrisected, as illustrated in FIG. 8. Then, according to setting of such brightness detection areas AS1 to AS5, since the brightness is detected in the entire area of the circular observation image outputted from the image generation portion 51, determination accuracy relating to whether or not the illumination light emitted through the emission end portion of the fiber 12 for illumination is intensively radiated to the minimum area outside the endoscope 2 can be improved.

In addition, according to the present embodiment, the brightness detection portion 52 may not only set the plurality of brightness detection areas in the observation image outputted from the image generation portion 51 but also set the entire area of the observation image as one brightness detection area BR, as illustrated in FIG. 9, for example. A specific example of the operation performed in such a case will be described below. Note that, hereinafter, specific description regarding parts to which the already-described operation or the like is applicable is appropriately omitted. FIG. 9 is a diagram illustrating one example of the brightness detection area set by the brightness detection portion.

The brightness detection portion 52 sets the entire area of the circular observation image for one frame outputted from the image generation portion 51 as one brightness detection area BR (see FIG. 9).

The brightness detection portion 52 acquires luminance values PB1 to PBN for N(2≦N) pixels which are all the pixels included in the brightness detection area BR (observation image) as the brightness detection value of the brightness detection area BR. In addition, the brightness detection portion 52 performs an arithmetic operation for acquiring an average luminance value BV which is the average of the luminance values PB1 to PBN, for example, as the brightness detection value of the brightness detection area BR. Then, the brightness detection portion 52 outputs the luminance values PB1 to PBN and the average luminance value BV acquired respectively as the brightness detection value of the brightness detection area BR to the determination portion 53.

The determination portion 53 acquires the judgement result by determining whether or not the brightness of the observation image generated by the image generation portion 51 is extreme brightness, based on the average luminance value BV which is the brightness detection value outputted from the brightness detection portion 52, and outputs the acquired judgement result to the light source control portion 25a.

More specifically, the determination portion 53 acquires the judgement result that the brightness of the observation image generated by the image generation portion 51 is extremely bright, and outputs the acquired judgement result to the light source control portion 25a, in the case of detecting that the average luminance value BV is larger than an upper limit luminance value TMAX, for example. In addition, the determination portion 53 acquires the judgement result that the brightness of the observation image generated by the image generation portion 51 is extremely dark, and outputs the acquired judgement result to the light source control portion 25a, in the case of detecting that the average luminance value BV is smaller than a lower limit luminance value TMIN, for example. Furthermore, the determination portion 53 acquires the judgement result that the brightness of the observation image generated by the image generation portion 51 is not the extreme brightness, and outputs the acquired judgement result to the light source control portion 25a, in the case of detecting that the average luminance value BV is within a predetermined range equal to or smaller than the upper limit luminance value TMAX and equal to or larger than the lower limit luminance value TMIN, for example. Note that the upper limit luminance value TMAX may be set as a luminance value in the case that halation occurs accompanying an approach of the distal end face of the endoscope 2 and the surface of the object, for example. In addition, the lower limit luminance value TMIN may be set as a luminance value in the case that the return light of a sufficient light quantity cannot be received accompanying separation of the distal end face of the endoscope 2 and the surface of the object, for example.

The determination portion 53 calculates the brightness dispersion amount based on the luminance values PB1 to PBN which are the brightness detection value outputted from the brightness detection portion 52, acquires the judgement result by determining the magnitude of the calculated brightness dispersion amount, and outputs the acquired judgement result to the light source control portion 25a.

More specifically, the determination portion 53 calculates variance VB of the luminance values PB1 to PBN outputted from the brightness detection portion 52 as the brightness dispersion amount, for example. Then, in the case of detecting that the value of the variance VB is smaller than a predetermined threshold THB, the determination portion 53 acquires the judgement result that the dispersion of the brightness in the observation image generated by the image generation portion 51 is small, and outputs the acquired judgement result to the light source control portion 25a. In addition, in the case of detecting that the value of the variance VB is equal to or larger than the predetermined threshold THB, the determination portion 53 acquires the judgement result that the dispersion of the brightness in the observation image generated by the image generation portion 51 is large, and outputs the acquired judgement result to the light source control portion 25a. Note that the predetermined threshold THB may be set according to the magnitude of a noise component that may be included in the photodetection signal outputted from the detection unit 23 to the image generation portion 51, for example.

That is, according to the operation of the determination portion 53 as described above, based on the magnitude of the value of the variance VB and the average luminance value BV, whether or not the illumination light emitted through the emission end portion of the fiber 12 for illumination is intensively radiated to the minimum area outside the endoscope 2 is determined. In addition, according to the operation of the determination portion 53 as described above, when it is detected that the average luminance value BV is within the predetermined range equal to or smaller than the upper limit luminance value TMAX and equal to or larger than the lower limit luminance value TMIN and the value of the variance VB is smaller than the predetermined threshold THB together, the judgement result indicating that the illumination light emitted through the emission end portion of the fiber 12 for illumination is intensively radiated to the minimum area outside the endoscope 2 is acquired. Furthermore, according to the operation of the determination portion 53 as described above, when it is detected that the average luminance value BV is larger than the upper limit luminance value TMAX, the average luminance value BV is smaller than the lower limit luminance value TMIN, or the value of the variance VB is equal to or larger than the predetermined threshold THB, the judgement result indicating that the illumination light emitted through the emission end portion of the fiber 12 for illumination is normally radiated to the object outside the endoscope 2 is acquired.

The light source control portion 25a performs the control for causing the light quantity of the illumination light emitted from the light source unit 21 to be maintained at the light quantity AL1 (present light quantity), in the case of detecting that the brightness of the observation image generated by the image generation portion 51 is extremely bright or the brightness of the observation image generated by the image generation portion 51 is extremely dark, for example, based on the judgement result outputted from the determination portion 53. In addition, the light source control portion 25a performs the control for causing the light quantity of the illumination light emitted from the light source unit 21 to be maintained at the light quantity AL1 (present light quantity), in the case of detecting that the brightness of the observation image generated by the image generation portion 51 is not the extreme brightness and the dispersion of the brightness in the observation image is large, for example, based on the judgement result outputted from the determination portion 53. Furthermore, the light source control portion 25a performs the control for causing the light quantity of the illumination light emitted from the light source unit 21 to be lowered to the light quantity AL2 lower than the light quantity AL1 (present light quantity), in the case of detecting that the brightness of the observation image generated by the image generation portion 51 is not the extreme brightness and the dispersion of the brightness in the observation image is small, for example, based on the judgement result outputted from the determination portion 53.

Therefore, even in the case that the entire area of the observation image outputted from the image generation portion 51 is set as one brightness detection area, effects similar to the effects in the case that the plurality of brightness detection areas are set in the observation image can be demonstrated.

On the other hand, the brightness detection portion 52 of the present embodiment may include a storage portion such as a RAM (random access memory) capable of storing a signal value of the photodetection signal successively outputted from the detection unit 23 in units of one frame, for example. In addition, in the case of including the above-described storage portion, the brightness detection portion 52 may, for example, instead of setting one or more brightness detection areas and acquiring the brightness detection value, acquire the signal value for one frame stored in the storage portion as luminance values PC1 to PCM for M(2≦M) pixels which are all the pixels included in the observation image for one frame generated by the image generation portion 51, acquire an average luminance value CV which is the average of the luminance values PC1 to PCM, and output the luminance values PC1 to PCM and the average luminance value CV to the determination portion 53. Note that, in such a case, for example, the determination portion 53 may calculate variance VC of the luminance values PC1 to PCM, and determine whether or not the illumination light emitted through the emission end portion of the fiber 12 for illumination is intensively radiated to the minimum area outside the endoscope 2, based on the magnitude of the value of the calculated variance VC and the average luminance value CV. Then, in the case that such determination is made in the determination portion 53, the judgement result almost similar to the judgement result when the brightness detection area BR is set is acquired.

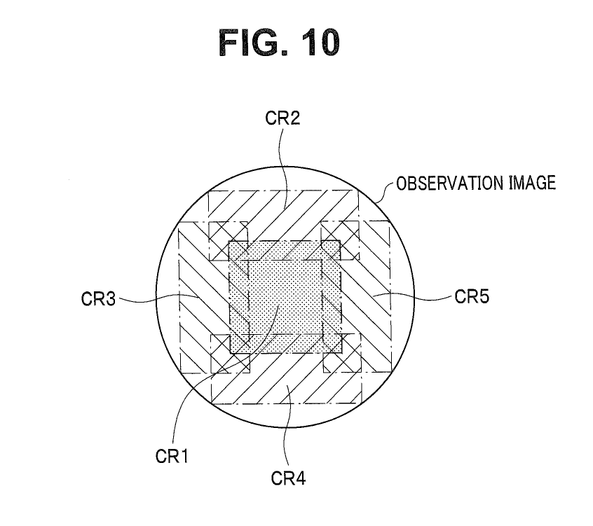

In addition, according to the present embodiment, the brightness detection portion 52 may not only set the brightness detection areas AR1 to ARS as illustrated in FIG. 7 but also set brightness detection areas CR1 to CR5 having a comparable area with each other, as illustrated in FIG. 10, for example. FIG. 10 is a diagram illustrating one example of the brightness detection area set by the brightness detection portion.

More specifically, the brightness detection area CR1 is set as a square area centering on the center pixel of the circular observation image outputted from the image generation portion 51, positioned more on the inner side than the outermost periphery of the circular observation image and including an overlapping part with each of the four brightness detection areas CR2 to CR5, as illustrated in FIG. 10. In addition, the brightness detection areas CR2 and CR4 are set respectively as a rectangular area including the overlapping part with each of the three brightness detection areas CR1, CR3 and CR5, being in contact with the outermost periphery of the circular observation image outputted from the image generation portion 51 and facing each other across the center pixel of the circular observation image, as illustrated in FIG. 10. Furthermore, the brightness detection areas CR3 and CR5 are set respectively as a rectangular area including the overlapping part with each of the three brightness detection areas CR1, CR2 and CR4, being in contact with the outermost periphery of the circular observation image outputted from the image generation portion 51 and facing each other across the center pixel of the circular observation image, as illustrated in FIG. 10. Then, according to the setting of such brightness detection areas CR1 to CR5, for example, the light adjusting control of suppressing occurrence of the halation in the observation image while preventing the brightness of a high luminance area of the observation image generated by the image generation portion 51 from being lowered as much as possible can be suitably performed.

In addition, according to the present embodiment, for example, in the case that the mapping table used in generation of the observation image by the image generation portion 51 is expressed by a predetermined function, a plurality of pieces of position data indicating the irradiation position of the illumination light when a predetermined object is scanned by the endoscope 2 may be stored in the memory 16 in a state of being converted to parameters applicable to the predetermined function.

Note that, for the present invention, regardless of the embodiment described above, it is needless to say that various changes and applications are possible without deviating from the gist of the invention.

What is claimed is:

1. An endoscope processor used in combination with a scanning type endoscope capable of scanning an object by swinging an optical fiber that transmits illumination light supplied from a light source portion and displacing an irradiation position of the illumination light, the endoscope processor comprising:
a photodetection portion configured to detect return light from the object irradiated with the illumination light and generate and successively output a photodetection signal according to the detected return light;
an image generation portion configured to generate an observation image of the object based on the photodetection signal; and
a determination portion configured to determine whether or not the illumination light emitted through the optical fiber is intensively radiated to a minimum area, based on a magnitude of dispersion of brightness in the observation image.

2. The endoscope processor according to claim 1, further comprising
a brightness detection portion configured to set one or more brightness detection areas in the observation image and acquire a brightness detection value corresponding to the one or more brightness detection areas,
wherein the determination portion calculates a brightness dispersion amount which is a value indicating the magnitude of the dispersion of the brightness in the observation image based on the brightness detection value obtained by the brightness detection portion, and determines whether or not the illumination light emitted through the optical fiber is intensively radiated to the minimum area based on the brightness dispersion amount.

3. The endoscope processor according to claim 2,
wherein the brightness detection portion sets a plurality of brightness detection areas in the observation image, and acquires a plurality of average luminance values corresponding to each of the plurality of brightness detection areas as the brightness detection value, and
the determination portion calculates variance of the plurality of average luminance values as the brightness dispersion amount, and determines whether or not the illumination light emitted through the optical fiber is intensively radiated to the minimum area, based on a magnitude of the value of the variance.

4. The endoscope processor according to claim 3,
wherein the determination portion acquires a judgement result indicating that the illumination light emitted through the optical fiber is intensively radiated to the minimum area, in a case of detecting that the magnitude of the value of the variance is smaller than a predetermined threshold.

5. The endoscope processor according to claim 4,
wherein control for lowering a light quantity of the illumination light from a present light quantity is performed, when the judgement result is acquired by the determination portion.

6. The endoscope processor according to claim 4,
wherein an operation for reporting the judgement result is performed, when the judgement result is acquired by the determination portion.

7. The endoscope processor according to claim 2,
wherein the brightness detection portion sets an entire area of the observation image as one brightness detection area and acquires luminance values of all pixels included in the one brightness detection area and an average luminance value which is an average of the luminance values of all the pixels as the brightness detection value respectively, and
the determination portion calculates variance of the luminance values of all the pixels as the brightness dispersion amount, and determines whether or not the illumination light emitted through the optical fiber is intensively radiated to the minimum area, based on a magnitude of the value of the variance and the average luminance value.

8. The endoscope processor according to claim 7,
wherein the determination portion acquires a determination result indicating that the illumination light emitted through the optical fiber is intensively radiated to the minimum area, in a case of detecting that the average luminance value is within a predetermined range and the magnitude of the value of the variance is smaller than a predetermined threshold together.

9. The endoscope processor according to claim 8,
wherein control for lowering a light quantity of the illumination light from a present light quantity is performed, when the judgement result is acquired by the determination portion.

10. The endoscope processor according to claim 2,
wherein the brightness detection portion acquires luminance values of all pixels included in the observation image and an average luminance value which is an average of the luminance values of all the pixels respectively based on a signal value of the photodetection signal, instead of setting the one or more brightness detection areas and acquiring the brightness detection value, and
the determination portion calculates variance of the luminance values of all the pixels, and determines whether or not the illumination light emitted through the optical fiber is intensively radiated to the minimum area, based on a magnitude of the value of the variance and the average luminance value.

11. The endoscope processor according to claim 3,
wherein the determination portion acquires a determination result indicating that the illumination light emitted through the optical fiber is intensively radiated to the minimum area, in a case of detecting that a magnitude of a current of a drive signal supplied to an actuator portion for swinging the optical fiber does not fluctuate from a specific magnitude and the magnitude of the value of the variance is smaller than a predetermined threshold together.
